(19) United States
(12) Patent Application Publication
(10) Pub. No.: US 2007/0232921 A1
(43) Pub. Date: Oct. 4, 2007

(54) Transducer assembly having a wide field of view

(75) Inventor: Warren Lee, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/396,736

(22) Filed: Apr. 3, 2006

(51) Int. Cl. H01L 41/00; A61B 8/00; A61B 8/14

(52) U.S. Cl. 600/459; 600/437; 310/311

(57) ABSTRACT

A transducer assembly is presented. The transducer assembly includes a cylindrical-shaped transducer array configured to transmit and receive acoustic energy over a three-dimensional volume. Additionally, the transducer assembly includes an acoustically absorbing shell disposed around the transducer array and configured to be rotatable with respect to the transducer array, wherein the acoustically absorbing shell is configured to selectively control directionality of acoustic energy transmitted and received by the transducer array.

### BACKGROUND

The invention relates generally to transducer assemblies, and more specifically to transducer assemblies for real-time imaging in space-constrained applications.

Catheter-based techniques used in interventional procedures generally involve inserting a probe, such as an imaging catheter, into a vein, such as the femoral vein. Unfortunately, many cardiac interventional procedures, such as ablation of atrial fibrillation, are complicated due to the lack of an efficient method to visualize interventional devices and cardiac anatomy in real-time. Intracardiac echocardiography (ICE) has recently gained interest as an emerging catheter imaging technology employed to guide interventional procedures, such as catheter positioning and ablation, for example.

Currently available catheter-based cardiac probes used for clinical ultrasound B-scan imaging suffer from limitations associated with the monoplanar nature of the B-scan images. Also, previously conceived solutions have incorporated one-dimensional catheter transducers to obtain three-dimensional images by rotating the entire catheter. However, the resulting images are not obtained in real-time. Additionally, mechanically scanning one-dimensional transducer arrays have been employed in relatively large probes where space constraints are not as severe.

Furthermore, previously conceived solutions for real-time three-dimensional intracardiac echocardiography employ two-dimensional arrays to steer and focus the ultrasound beam over a pyramidal-shaped volume. Unfortunately, these two-dimensional arrays require a relatively large number of interconnections in order to adequately sample the acoustic aperture space to achieve sufficient spatial resolution and image quality, thereby resulting in poor space efficiency of the transducer assemblies. In addition, these probes suffer from other drawbacks such as poor imaging resolution, low sensitivity and increased system cost and complexity.

There is therefore a need for a design of a transducer assembly capable of two-dimensional imaging and/or real-time three-dimensional imaging for use in a probe employed in space-constrained applications such as intracardiac imaging. In particular, there is a significant need for a design of a transducer assembly having a relatively wide field of view, thereby resulting in enhanced image resolution and sensitivity of the probe. Also, it would be desirable to develop a simple and cost-effective method of fabricating a transducer assembly capable of real-time three-dimensional imaging.

### BRIEF DESCRIPTION

Briefly, in accordance with aspects of the technique, a transducer assembly is presented. The transducer assembly includes a cylindrical-shaped transducer array configured to transmit and receive acoustic energy over a three-dimensional volume. Additionally, the transducer assembly includes an acoustically absorbing shell disposed around the transducer array and configured to be rotatable with respect to the transducer array, where the acoustically absorbing shell is configured to selectively control directionality of acoustic energy transmitted and received by the transducer array.

In accordance with further aspects of the technique, an invasive probe configured to image an anatomical region is presented. The invasive probe includes an outer envelope sized and configured to be disposed in the anatomical region. Further, the invasive probe also includes a transducer assembly disposed in the outer envelope, where the transducer assembly includes a cylindrical-shaped transducer array configured to transmit and receive acoustic energy over a three-dimensional volume, and an acoustically absorbing shell disposed around the transducer array and configured to be rotatable with respect to the transducer array, where the acoustically absorbing shell is configured to selectively control directionality of acoustic energy transmitted and received by the transducer array.

In accordance with yet another aspect of the technique, a system is presented. The system includes an acquisition subsystem configured to acquire image data, where the acquisition subsystem includes an invasive probe configured to image an anatomical region, where the invasive probe includes an outer envelope sized and configured to be disposed in the anatomical region; and a transducer assembly disposed in the outer envelope, where the transducer assembly comprises a cylindrical-shaped transducer array configured to transmit and receive acoustic energy over a three-dimensional volume, and an acoustically absorbing shell disposed around the transducer array and configured to be rotatable with respect to the transducer array, where the acoustically absorbing shell is configured to selectively control directionality of acoustic energy transmitted and received by the transducer array. In addition, the system includes a processing subsystem in operative association with the acquisition subsystem and configured to process the image data acquired via the acquisition subsystem.

In accordance with further aspects of the technique, a method for imaging is presented. The method includes energizing a transducer array in a transducer assembly disposed in an invasive probe, where the transducer assembly includes an acoustically absorbing shell disposed around the transducer array and configured to be rotatable with respect to the transducer array, where the acoustically absorbing shell comprises an acoustic window. Also, the method includes selectively controlling directionality of acoustic energy transmitted by a portion of the transducer array aligned with the acoustic window.

### DRAWINGS

These and other features, aspects, and advantages of the invention will become better understood when the following detailed description is read with reference to the accompanying drawings in which like characters represent like parts throughout the drawings, wherein:

FIG. 1 is a block diagram of an exemplary ultrasound imaging and therapy system, in accordance with aspects of the present technique;

FIG. 2 is a perspective view of an exemplary embodiment of a transducer array for use in the system illustrated in FIG. 1, in accordance with aspects of the present technique;

FIG. 3 is a side view of a transducer element for use in the exemplary embodiment of the transducer array illustrated in FIG. 2, in accordance with aspects of the present technique;

FIG. 4 is a side view of another exemplary transducer element for use in a transducer array, in accordance with aspects of the present technique;

FIG. 5 is a diagram showing assembly of an exemplary embodiment of a transducer assembly including the transducer array illustrated in FIG. 2, in accordance with aspects of the present technique;

FIG. 6 is an illustration of an exemplary transducer assembly including the transducer array illustrated in FIG. 2, in accordance with aspects of the present technique;

FIG. 7 is an illustration of another exemplary transducer assembly including the transducer array illustrated in FIG. 2, in accordance with aspects of the present technique;

FIG. 8 is a schematic flow chart depicting an exemplary method for imaging employing the transducer assembly illustrated in FIG. 6, in accordance with aspects of the present technique;

FIG. 9 is a perspective view of an invasive probe including the transducer assembly illustrated in FIG. 6, in accordance with aspects of the present technique; and

FIG. 10 is an illustration of imaging and delivering therapy employing the exemplary transducer assembly illustrated in FIG. 6, in accordance with aspects of the present technique.

### DETAILED DESCRIPTION

As will be described in detail hereinafter, a transducer assembly capable of real-time three-dimensional imaging and configured for use in an invasive probe employed in space-constrained applications, such as intracardiac imaging, and methods of imaging are presented. Employing the invasive probe having the exemplary transducer assembly a relatively wide three-dimensional field of view may be obtained. Based on the image data acquired by the invasive prove, a user may assess need for therapy in an anatomical region and direct the therapy via the invasive probe.

Although, the exemplary embodiments illustrated hereinafter are described in the context of a medical imaging system, it will be appreciated that use of the transducer assembly with a wide field of view in industrial applications are also contemplated in conjunction with the present technique.

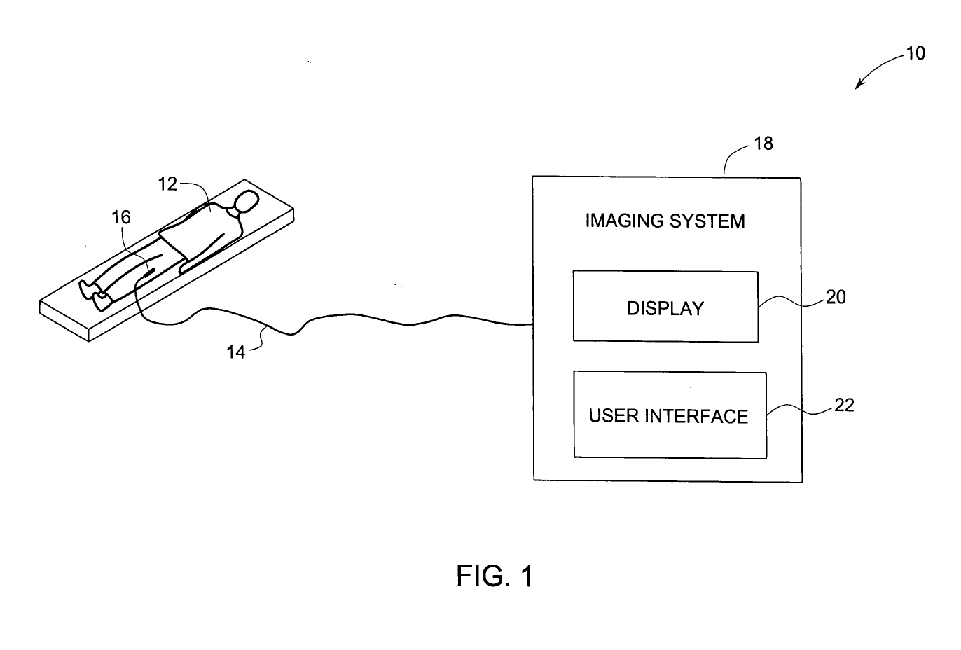

FIG. 1 is a block diagram of an exemplary system 10 for use in imaging, in accordance with aspects of the present technique. As will be appreciated by one skilled in the art, the figures are for illustrative purposes and are not drawn to scale. The system 10 may be configured to facilitate acquisition of image data from a patient 12 via a probe 14. In other words, the probe 14 may be configured to acquire image data representative of a region of interest in the patient 12, for example. In accordance with aspects of the present technique, the probe 14 may be configured to facilitate interventional procedures. In other words, in a presently contemplated configuration, the probe 14 may include an invasive probe. It should also be noted that, although the embodiments illustrated are described in the context of a catheter-based probe, other types of probes such as endoscopes, laparoscopes, surgical probes, transrectal probes, transvaginal probes, intracavity probes, probes adapted for interventional procedures, or combinations thereof are also contemplated in conjunction with the present technique. Reference numeral 16 is representative of a portion of the probe 14 disposed inside the patient 12. In certain embodiments, the probe may include an imaging catheter-based probe 14. The imaging catheter 14 may include a real-time imaging transducer assembly having a relatively wide field of view (not shown).

The system 10 may also include an imaging system 18 that is in operative association with the imaging catheter 14 and configured to facilitate acquisition of image data. It should be noted that although the exemplary embodiments illustrated hereinafter are described in the context of a medical imaging system, such as an ultrasound system, other imaging systems, such as, but not limited to, optical imaging systems, pipeline inspection systems, liquid reactor inspection systems, or other imaging systems are also contemplated. In addition, although the embodiments illustrated hereinafter are described in the context of an ultrasound imaging system, other medical imaging systems, such as, but not limited to, optical coherence tomography are also envisaged.

Further, the imaging system 18 may be configured to display an image representative of a current position of the probe 14 within a region of interest in the patient 12. As illustrated in FIG. 1, the imaging system 18 may include a display area 20 and a user interface area 22. In accordance with aspects of the present technique, the display area 20 of the imaging system 18 may be configured to display the image generated by the imaging system 18 based on the image data acquired via the probe 14. Additionally, the display area 20 may be configured to aid the user in visualizing the generated image.

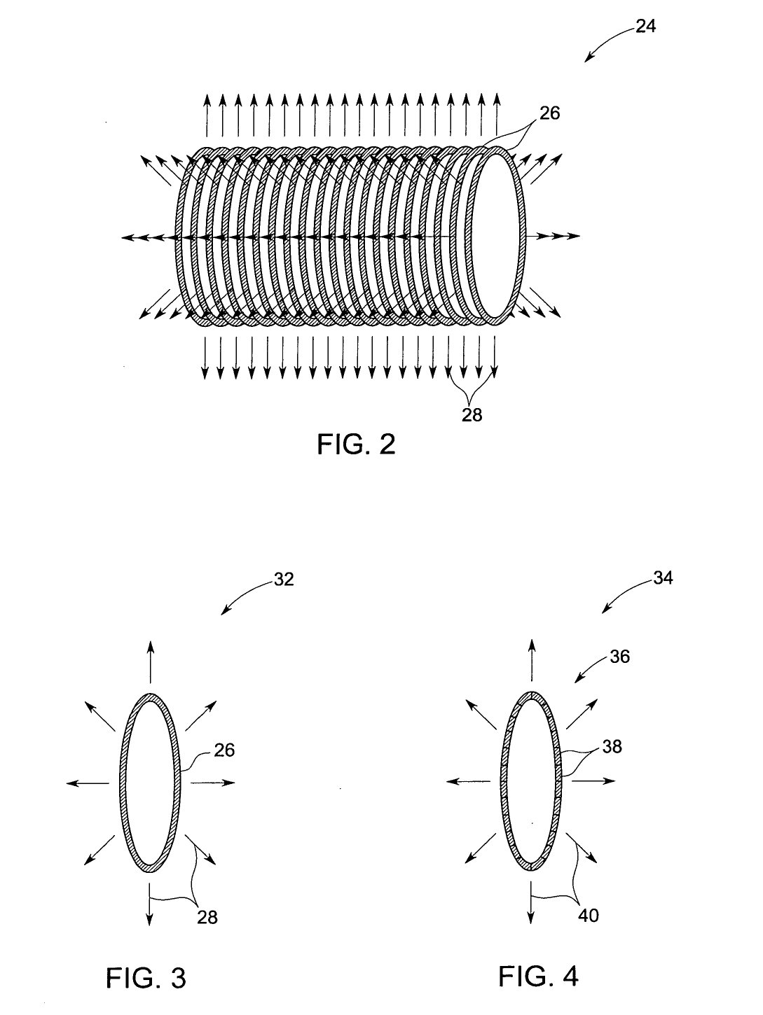

FIG. 2 is an illustration of an exemplary embodiment 24 of a cylindrical-shaped transducer array with ring-shaped elements for use in the system 10 illustrated in FIG. 1. The embodiment of the transducer array 24 illustrated in FIG. 2 is shown as having a plurality of ring-shaped transducer elements 26. In a presently contemplated configuration, each of the plurality of transducer elements 26 may include a single, continuous ring-shaped element. Moreover, each of the plurality of transducer elements 26 may be configured to transmit and receive acoustic energy over a three-dimensional volume. In one embodiment, the three-dimensional volume may include a cylindrical-shaped three-dimensional volume. Reference numeral 28 is representative of the acoustic energy transmitted by the transducer elements 26. It may also be noted that each of the plurality of transducer elements 26 in the transducer array 24 may be configured to be individually addressable by operatively coupling each of the plurality of transducer elements 26 to a respective system channel (not shown). Furthermore, the transducer array 24 may be configured to fit within a probe as will be described in greater detail hereinafter.

FIG. 3 illustrates a side view 32 of a ring-shaped transducer element, such as the transducer element 26 (see FIG. 2). The illustrated ring-shaped transducer element 26 may be configured to transmit and receive acoustic energy throughout its circumference and centered about a plane of the transducer element 26. Although the exemplary embodiment of the transducer elements 26 illustrated are described in the context of a ring-shaped transducer element, other shapes, such as, but not limited to, an oval-shaped transducer element or an elliptical-shaped transducer element are also contemplated in conjunction with the present technique.

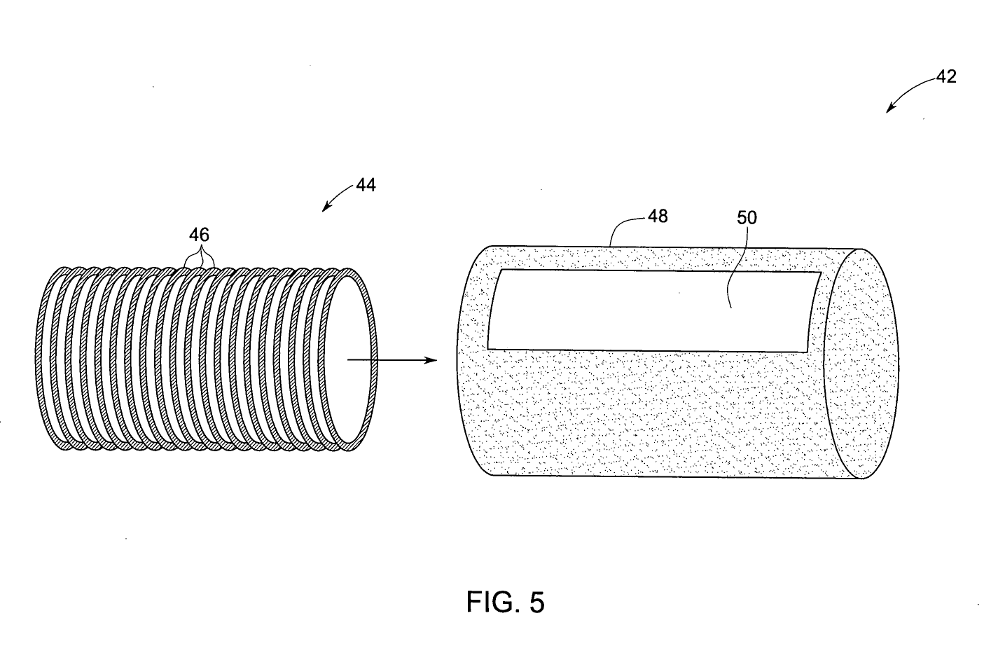

Turning now to FIG. 4, an alternate embodiment 34 of a ring-shaped transducer element 36 for use in the transducer array 24 (see FIG. 2) is illustrated. In this embodiment, the transducer element 36 may be formed by operatively coupling a plurality of segments 38 in series to form a ring. Also, reference numeral 40 is representative of acoustic energy transmitted by the ring-shaped transducer element 36.

In accordance with aspects of the present technique, the transducer array 24 may include a micromachined ultrasound array, a lead zirconate array or combinations thereof. Alternatively, each of the plurality of ring-shaped elements may be formed from a piezocomposite material. As will be appreciated, piezocomposite materials are typically made of thin rods of ceramics embedded into a polymer material. Further, piezocomposite materials have been know to have a high coupling coefficient that confers to the transducers a high sensitivity and signal to noise ratio. Piezocomposite materials also exhibit a higher mechanical resistance that confers to the transducers a higher resistance to mechanical shocks, vibrations, temperature constraints and pressure constraints. Additionally, the piezocomposite materials may be mechanically focused, which advantageously allows the manufacturing of cylindrical, spherical or curved transducers.

FIG. 5 illustrates assembly 42 of an exemplary embodiment of a transducer assembly. Reference numeral 44 is representative of a cylindrical-shaped transducer array, such as the cylindrical-shaped transducer array 24 (see FIG. 2). As previously noted, the cylindrical-shaped transducer array 44 may include a plurality of ring-shaped transducer elements 46. Also, since the transducer array 44 is configured to transmit and receive acoustic energy throughout its circumference and centered about the respective plane of each of the plurality of transducer elements 46, it may be desirable to control the directionality of the acoustic energy transmitted and/or received in order to assemble meaningful volumetric data. Accordingly, an acoustically absorbing shell 48 configured to control the directionality of the acoustic energy transmitted and/or received by the transducer array 44 is presented.

Following assembly of the transducer array 44, the acoustically absorbing shell 48 may be disposed around the transducer array 44, in accordance with exemplary aspects of the present technique. The acoustically absorbing 48 may include a material that is configured to attenuate and/or absorb the acoustic energy transmitted by the transducer array 44. In other words, the acoustically absorbing shell 48 may be configured to facilitate attenuation and/or absorption of acoustic energy that may emerge from the transducer elements 46. In one embodiment, the acoustically absorbing shell 48 may include a cylindrical shape. Furthermore, in accordance with exemplary embodiments of the present technique, the transducer array 44 may be configured to be substantially stationary, while the acoustically absorbing shell 48 may be configured to rotate with respect to the transducer array 44.

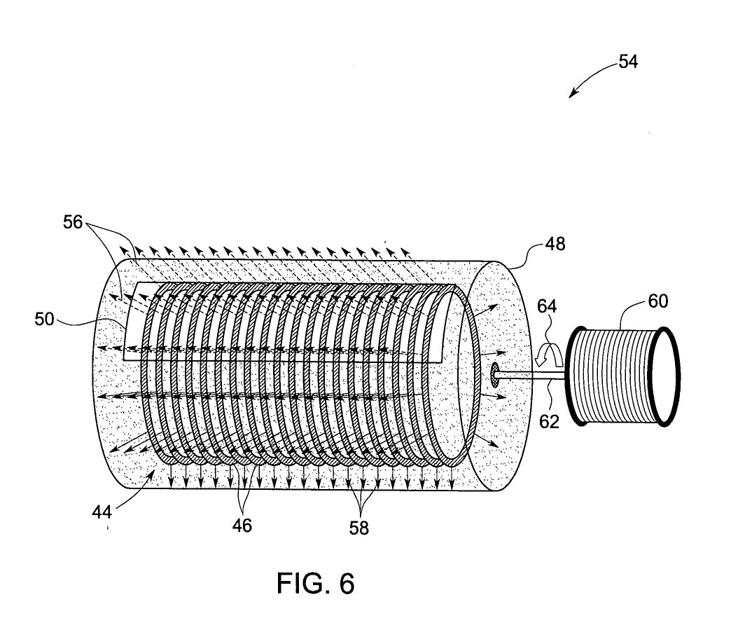

Furthermore, the acoustically absorbing shell 48 may include an acoustic window 50 that is at least partially transparent to the acoustic energy transmitted and/or received by the transducer array 44. The acoustic window 50 may be configured to selectively control the directionality of the acoustic energy. As used herein, “to selectively control directionality” refers to guiding the transmission and reception of acoustic energy about the transducer array 44. In other words, acoustic energy transmitted by a select portion of the transducer array 44 may be directed at an object of interest via the acoustic window 50, while the acoustic energy transmitted by the other portions of the transducer array 44 may be attenuated and absorbed by the acoustically absorbing shell 48. In a similar fashion, acoustic energy reflected by the object of interest may be received by a select portion of the transducer array 44 via the acoustic window 50. In other words, the acoustic energy transmitted by transducer array 44 may be directed toward an object of interest, such as the patient 12 (see FIG. 1), via the acoustic window 50. In a similar fashion, the acoustic window 50 may be configured to facilitate reception of acoustic energy by the transducer array 44. Accordingly, the acoustic energy may only pass through the acoustic window 50. The functioning of the transducer assembly 44 will be described in greater detail with respect to FIG. 6.

Referring now to FIG. 6, a perspective view 54 of an exemplary transducer assembly is illustrated. In the illustrated embodiment, the acoustically absorbing shell 48 is disposed about the cylindrical-shaped transducer array 44. In accordance with aspects of the present technique, the transducer assembly 54 may also include a motor 60. The motor 60 may be operatively coupled to the acoustically absorbing shell 48. In one embodiment, the motor 60 may be operatively coupled to the acoustically absorbing shell 48 via a drive shaft 62. Furthermore, the motor 60 may be configured to rotate the acoustically absorbing shell 48 about the transducer array 44 such that the acoustic window 50 is oriented to selectively control the directionality of the acoustic energy transmitted and/or received by the transducer array 44. In other words, the motor 60 may be configured to facilitate rotating the acoustic window 50 in the acoustically absorbing shell 48 in order to vary the direction of the acoustic energy transmitted and/or received by the transducer array 44. Reference numeral 64 is representative of a direction of rotation of the acoustically absorbing shell 48.

In accordance with aspects of the present technique, the motor 60 may be configured to rotate the acoustically absorbing shell 48 in a continuous mode, an oscillation mode or combinations thereof. It may be noted that rotating the acoustically absorbing shell 48 in a continuous mode would advantageously facilitate obtaining a 360-degree field of view. Further, the acoustically absorbing shell 48 may be rotated in an oscillating mode when a field of view of less than about 360 degrees is desired. Accordingly, the oscillating mode of rotating the acoustically absorbing shell 48 may be employed to facilitate an increase in the frame rate of a select portion of the 360-degree field of view.

As previously noted, the cylindrical-shaped transducer array 44 is configured to transmit and/or receive acoustic energy throughout the circumference of the transducer array 44. However, the acoustically absorbing shell 48 may be configured to attenuate and/or absorb any acoustic energy impinging thereon. The acoustic energy that is attenuated and absorbed by the acoustically absorbing shell 48 is represented generally by reference numeral 58. Additionally, the acoustic window 50 may also be configured to allow passage of the acoustic energy transmitted by the transducer array 44. The acoustic energy that is transmitted towards the object of interest via the acoustic window 50 is generally represented by reference numeral 56. It may be noted that means for acoustic coupling may be disposed between the transducer array 44 and the acoustically absorbing shell 48. The acoustic coupling means may be configured to couple the acoustic energy from the transducer array 44 to the acoustically absorbing shell 48. In certain embodiments, the acoustic coupling means may include an acoustic coupling fluid or a gel, for example.

Additionally, the transducer assembly 54 may include an interconnect layer (not shown). The interconnect layer may include a flexible interconnect layer, in certain embodiments. Further, the flexible interconnect layer may include at least one conductive element disposed on a flexible substrate. As will be appreciated, the at least one conductive element may be configured to facilitate coupling each of the plurality of transducer elements 46 in the transducer array 44 to a cable assembly or electronics, for example. In other words, the interconnect layer may be configured to be in operative association with the transducer array 44 on one end and a cable assembly (not shown) or electronics (not shown) at the other end.

As previously noted, the transducer array 44 may be configured to remain substantially stationary while the acoustically absorbing shell 48 may be configured to rotate with respect to the transducer array 44. As will be appreciated by one skilled in the art, need for rotating interconnect coupled to a rotating transducer array disadvantageously results in increased torque requirements on the motor 60. By implementing the transducer assembly 54 having a substantially stationary transducer array 44 and a rotating acoustically absorbing shell 48 as described hereinabove, the need for rotating associated interconnect may advantageously be circumvented. Consequently, torque requirements on the motor 60 may advantageously be minimized as the motor 60 is configured to rotate only the acoustically absorbing shell 48 and not the interconnect.

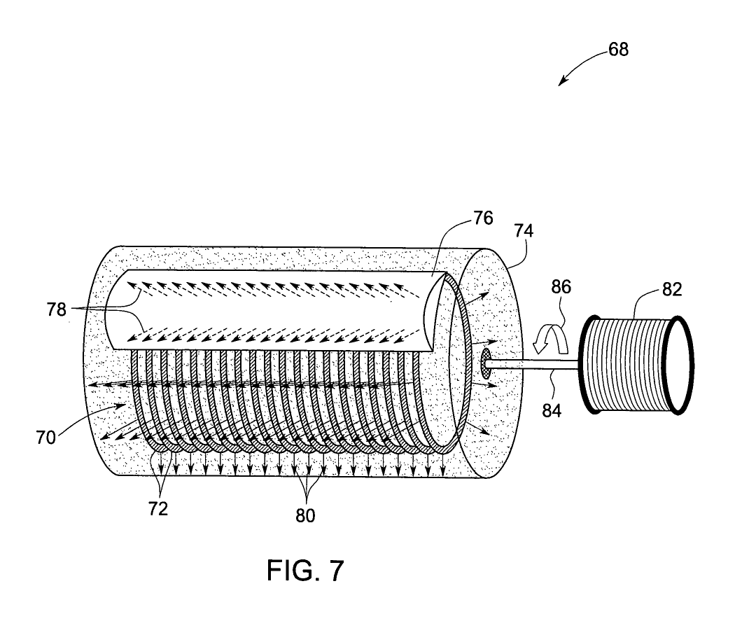

Turning now to FIG. 7, another exemplary embodiment 68 of a transducer assembly is illustrated. In this embodiment, the transducer assembly 68 is shown as including a cylindrical-shaped transducer array 70, where the cylindrical-shaped transducer array 70 may include a plurality of ring-shaped transducer elements 72. In addition, an acoustically absorbing shell 74 may be disposed about the transducer array 70. As previously noted, the acoustically absorbing shell 74 may include an acoustic window (not shown) configured to selectively control the directionality of the acoustic energy transmitted and/or received by the transducer array 70. In accordance with aspects of the present technique, the transducer assembly 68 may also include a focusing lens 76. The focusing lens 76 may be disposed such that the focusing lens substantially covers the acoustic window on the acoustically absorbing shell 74. Further, the focusing lens 76 may be configured to facilitate focusing the transmission and/or reception of acoustic energy by the transducer array 70 through the acoustic window.

Energy that passes through the lens 76 is represented by reference numeral 78, while reference numeral 80 is representative of the acoustic energy that is attenuated and/or absorbed by the acoustically absorbing shell 74. In addition, the transducer assembly 68 may include a motor 82 that may be- operatively coupled to the acoustically absorbing shell 74 via a drive shaft 84, where the motor 82 is configured to rotate the acoustically absorbing shell 74 about the transducer array 70. A direction of rotation of the drive shaft 84 is generally represented by reference numeral 86.

The embodiments of the transducer assembly 54, 68 illustrated in FIGS. 6-7 respectively may accordingly be of a size or dimension suitable for use in an invasive probe employed in space-constrained applications. In certain embodiments, the invasive probe may include an imaging catheter, an endoscope, a laparoscope, a surgical probe, a transesophageal probe, a transvaginal probe, a transrectal probe, an intracavity probe, or a probe adapted for interventional procedures, as previously noted.

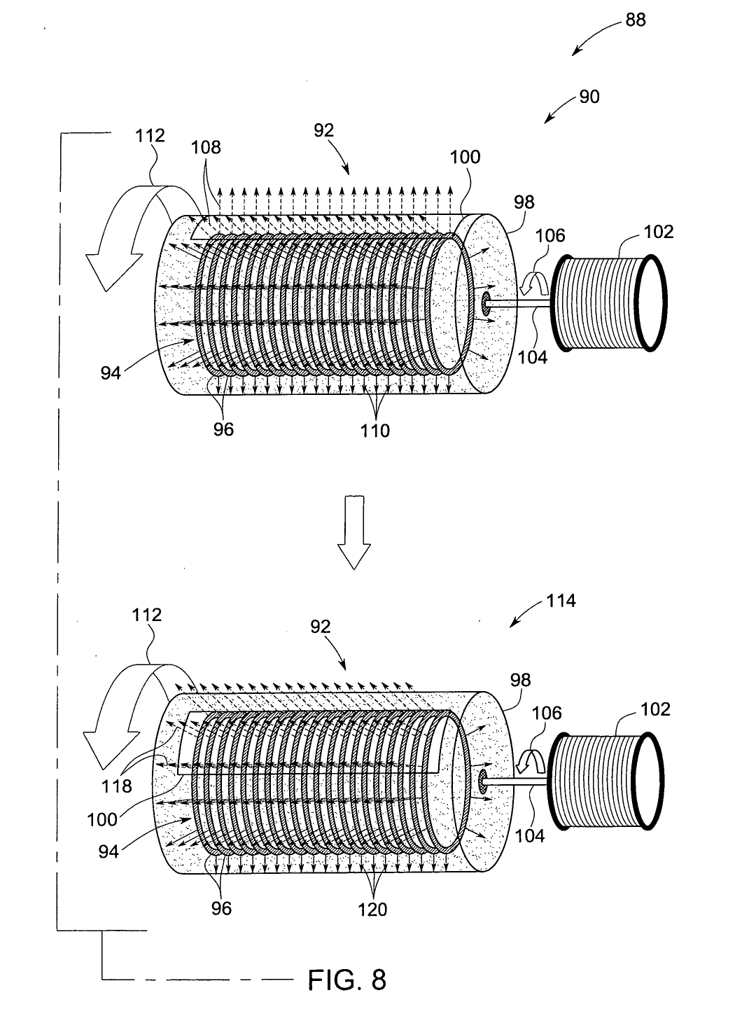

FIG. 8 illustrates an exemplary method of imaging 88 employing a transducer assembly, such as the transducer assembly 54 illustrated in FIG. 6. In the illustrated embodiment, reference numeral 90 is representative of a transducer assembly 92 with an acoustic window 100 oriented in a first position. In the illustrated embodiment 90, the transducer assembly 92 is shown as including a cylindrical-shaped transducer array 94, where the cylindrical-shaped transducer array 94 may include a plurality of ring-shaped transducer elements 96. Also, an acoustically absorbing shell 98 may be disposed about the transducer array 94, where the acoustically absorbing shell 98 may be configured to attenuate and/or absorb acoustic energy transmitted by the transducer array 94. Furthermore, the acoustically absorbing shell 98 may include an acoustic window 100 configured to selectively control the directionality of the acoustic energy transmitted and/or received by the transducer array 94, as previously noted. Additionally, a motor 102 may be operatively coupled to the acoustically absorbing shell 98 via a drive shaft 104, for example. As previously noted, the motor 102 may be configured to facilitate rotating the acoustically absorbing shell 98 with respect to the transducer array 94. Reference numeral 106 is representative of a direction of rotation of the drive shaft 104.

The acoustic window 100 may be employed to selectively control the directionality of the acoustic energy transmitted and/or received by transducer array 94. Accordingly, the motor 102 may be configured to rotate the acoustically absorbing shell 98 such that the acoustic window 100 is oriented at a plurality of positions with respect to the transducer array 94. Consequently, the directionality of the acoustic energy may be determined by the position of the acoustic window 100. The method of imaging employing the exemplary transducer assembly 92 is described hereinafter.

The plurality of transducer elements 96 in the transducer array 94 may be energized to facilitate generation of acoustic energy by the plurality of transducer elements 96. Subsequently, the directionality of the acoustic energy transmitted by the transducer array 94 may be selectively controlled. In other words, the acoustically absorbing shell 98 may be rotated about the transducer array 94 such that the acoustic window 100 is oriented at a plurality of positions to selectively control directionality of the acoustic energy transmitted and/or received by the transducer array 94 via the acoustic window 100. As previously noted, the motor 102 that is in operative association with the acoustically absorbing shell 98 may be employed to rotate the acoustically absorbing shell 98. Accordingly, the acoustically absorbing shell 98 may be rotated such that the acoustic window 100 is oriented in a first position as illustrated in the embodiment 92 of the transducer assembly.

With the acoustic window 100 oriented in the first position, acoustic energy 108 transmitted generated by a portion of the transducer array 94 that is aligned with the acoustic window 100 in the first position may be directed at the object of interest via the acoustic window 100. Further, acoustic energy 110 generated by portions of the transducer array 94 that are presently not aligned with the acoustic window 100 oriented in the first position may be attenuated and/or absorbed by the acoustically absorbing shell 98, as previously described. In a similar fashion, acoustic energy reflected from the object may be received by the same portion of the transducer array 94 that is presently aligned with the acoustic window 100 oriented in the first position. Consequently, a first single scan plane may be generated. It may be noted that the transducer array 94 may be operated as a sector phased array, a linear sequential array, or any conventional scanning method. Reference numeral 112 represents generally a direction of rotation of the acoustically absorbing shell 98.

Subsequently, the acoustically absorbing shell 98 may be further rotated to orient the acoustic window in a second position as illustrated in the embodiment 114 of the transducer assembly 92. In other words, reference numeral 114 is representative of the transducer assembly 92 with the acoustic window 100 oriented in a second position. As described hereinabove with reference to the transducer assembly 92 in the first position, acoustic energy 118 transmitted by a portion of the transducer array 94 presently aligned with the acoustic window 100 oriented in the second position may be directed towards the object of interest. Energy reflected from the object of interest may then be received by the portion of the transducer array 94 that is currently aligned with the acoustic window 100 oriented in the second position. Consequent to the transmission of acoustic energy 118 towards the object of interest and subsequent reception of acoustic energy by the portion of the transducer array 94 presently in alignment with the acoustic window 100 oriented in the second position, a second single scan plane may be generated. Reference numeral 120 is representative of acoustic energy that is attenuated and/or absorbed by the acoustically absorbing shell 98.

As described hereinabove, a single scan plane may be generated for each of the positions of the acoustic window 100. Accordingly, the acoustically absorbing shell 98 may be rotated with respect to the transducer array 94 such that the acoustic window 100 is oriented at a plurality of positions. A respective single scan plane may then be generated at each of these positions of the acoustic window 100. These single scan planes may then be assembled to obtain volumetric image data having a relatively wide field of view. In one embodiment, the volumetric image data having a relatively wide field of view may include image data in a range from about 10 degrees to about 360 degrees. Accordingly, volumetric image data may be obtained by rotating the acoustically absorbing shell 98 about the transducer array 94 such that the acoustic window 100 is oriented at a plurality of positions to facilitate acquisition of a plurality of scan planes that may then be assembled to obtain a volumetric or partial volumetric image of a given region of interest. As previously noted, the motor 102 may be configured to rotate the acoustically absorbing shell 98 in a continuous mode, an oscillation mode, or combinations thereof. In addition, the motor 102 may be configured to rotate the acoustically absorbing shell 98 such that the acoustic window 100 is oriented at a plurality of positions about the circumference of the transducer array 94. Alternatively, the acoustically absorbing shell 98 may be rotated such that the acoustic window 100 is oriented at a plurality of positions through a predetermined part of the circumference of the transducer array 94.

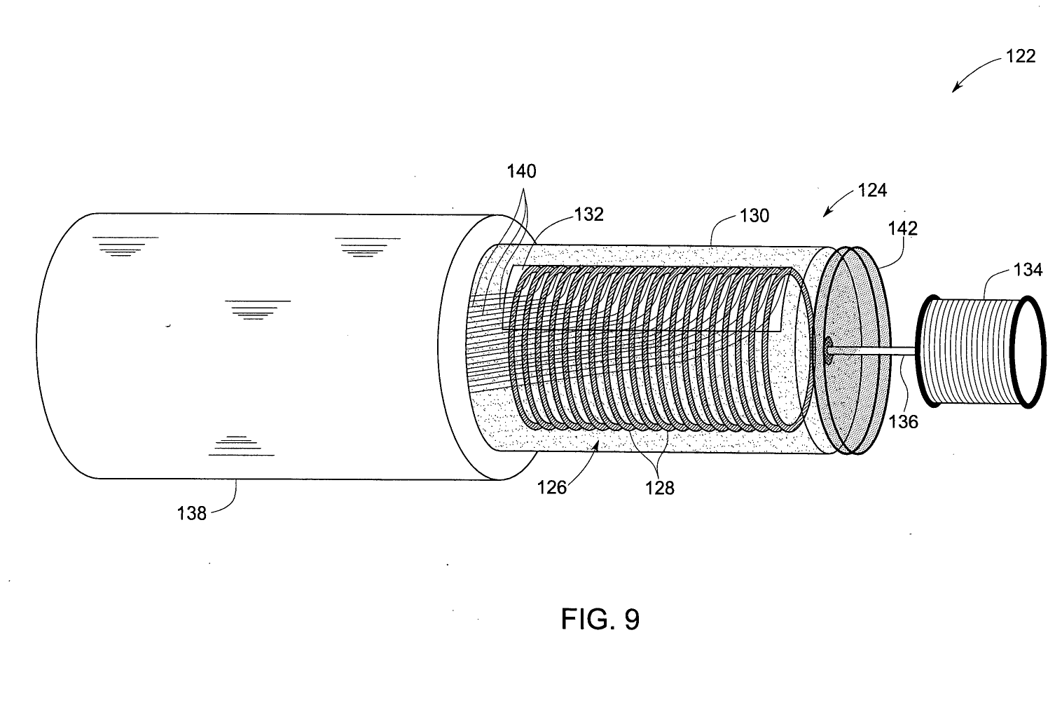

FIG. 9 illustrates an exemplary embodiment 122 of an invasive probe having a transducer assembly 124, such as the transducer assembly depicted in FIGS. 6-7. According to aspects of the present technique, the invasive probe 122 may be configured to facilitate imaging an anatomical region in space-constrained applications. In certain embodiments, the invasive probe 122 may include an imaging catheter, an endoscope, a laparoscope, a surgical probe, a transesophageal probe, a transvaginal probe, a transrectal probe, an intracavity probe, or a probe adapted for interventional procedures, as previously noted.

The transducer assembly 124 may be produced as previously described. Also, as previously noted, the transducer assembly 124 may be sized and configured to fit inside an invasive probe configured for use in space-constrained applications, such as cardiac imaging. The transducer assembly 124 may include a cylindrical-shaped transducer array 126, where the cylindrical-shaped transducer array 126 may include a plurality of ring-shaped transducer elements 128. Furthermore, an acoustically absorbing shell 130 having an acoustic window 132 may be disposed around the transducer array 126. The transducer assembly 124 may also include a motor 134 that is in operative association with the acoustically absorbing shell 130 via a drive shaft 136.

In one embodiment, the transducer assembly 124 may be disposed in an outer envelope 138 of an invasive probe 122 as illustrated in FIG. 9. In a presently contemplated configuration, the transducer assembly 124 may be disposed on a distal tip of an invasive probe 122. Reference numeral 140 is representative of interconnect configured to electrically couple the plurality of ring-shaped transducer elements 128 in the transducer array 126 to a cable assembly (not shown) or electronics (not shown), as previously described.

Additionally, the invasive probe 122 may also include a position sensor 142 that may be configured to monitor a position of the acoustic window 132. It may also be noted that in certain embodiments, the motor 134 may be disposed within the invasive probe 122. Alternatively, in certain other embodiments, the motor 134 may be disposed external to the invasive probe 122. In the embodiment of the invasive probe where the motor 134 is disposed outside the invasive probe 122 a relatively long drive shaft may be employed to operatively couple the motor 134 to the acoustically absorbing shell 130.

As previously noted with reference to FIG. 6, means for acoustic coupling (not shown) may be disposed between the transducer array 126 and the acoustically absorbing shell 130. The acoustic coupling means may be configured to couple the acoustic energy from the transducer array 126 to the acoustically absorbing shell 130. In certain embodiments, the acoustic coupling means may include an acoustic coupling fluid or a gel, as previously described. Additionally, means for acoustic coupling (not shown) may be disposed between the acoustically absorbing shell 130 and the outer envelope 138. The acoustic coupling means may be configured to couple the acoustic energy from the acoustically absorbing shell 130 to the outer envelope 138. In certain embodiments, the acoustic coupling means may include an acoustic coupling fluid or a gel, for example.

By implementing the invasive probe 122 having the transducer assembly 124 as described hereinabove, real-time three-dimensional image volumes having a relatively large field of view may be obtained by the transducer assembly 126 from within the invasive probe 122. These real-time, three-dimensional imaging volumes having a relatively wide field of view may then be advantageously employed to facilitate guidance of cardiac interventional procedures, for instance. In addition, the invasive probe 122 may be configured for space-constrained applications such as intracardiac echocardiography (ICE) and transesophageal echocardiography (TEE), for example. Furthermore, volumetric images may be obtained with minimal system complexity and channel count requirements, thereby allowing use of these probes in portable, miniaturized systems.

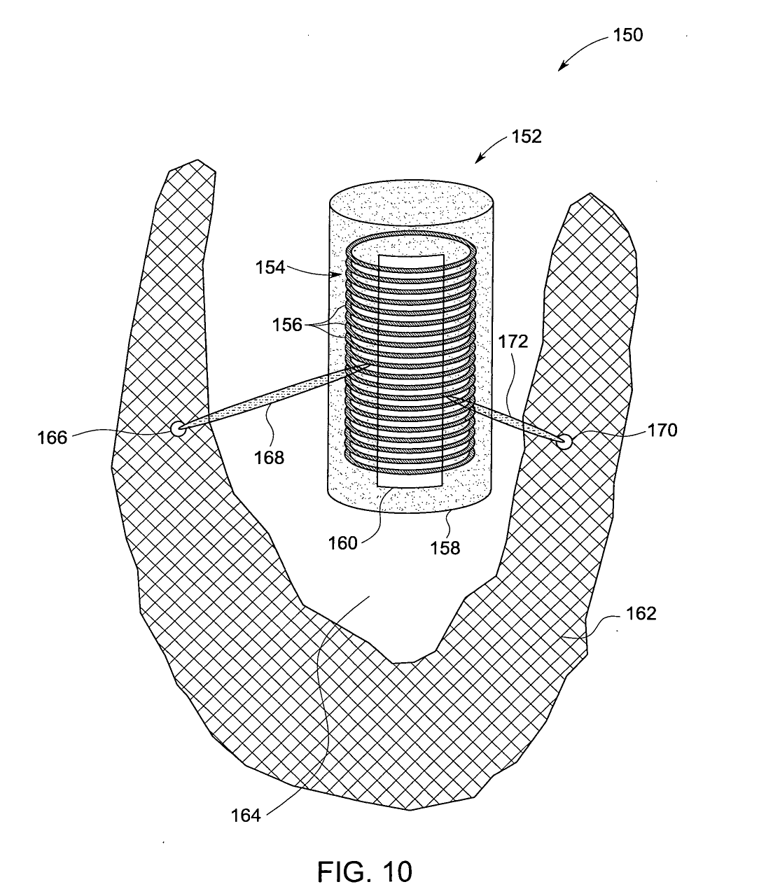

In accordance with exemplary aspects of the present technique, the invasive probe 122 may also be configured to facilitate delivering therapy to one or more regions of interest within the anatomical region in addition to imaging in space-constrained applications. FIG. 10 illustrates a method of imaging and delivering therapy employing the exemplary transducer assembly illustrated in FIG. 6, in accordance with aspects of the present technique. According to exemplary aspects of the present technique, the imaging aspects of the invasive probe described hereinabove may be advantageously coalesced with delivery of therapy to one or more regions of interest. As used herein, “therapy” is representative of ablation, percutaneous ethanol injection (PEI), cryotherapy, and laser-induced thermotherapy. Additionally, “therapy” may also include delivery of tools, such as needles for delivering gene therapy, for example. Additionally, as used herein, “delivering” may include various means of providing therapy to the one or more regions of interest, such as conveying therapy to the one or more regions of interest or directing therapy towards the one or more regions of interest. As will be appreciated, in certain embodiments, the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

As will be appreciated, catheter-based interventional procedures typically involve inserting the invasive probe into a vein, such as the femoral vein. The invasive probe may then be guided from the point of entry, through the vasculature of a patient to a desirable anatomical location, such as the heart, for example. In the illustrated embodiment 150, the transducer assembly 152 is shown independent of an invasive probe (not shown), a motor (not shown) and a drive shaft (not shown) for simplicity of illustration. The transducer assembly 152 is illustrated as including a cylindrical-shaped transducer array 154, where the transducer array 154 may include a plurality of ring-shaped transducer elements 156, as previously described. Moreover, an acoustically absorbing shell 158 having an acoustic window 160 configured to selectively control the directionality of acoustic energy transmitted and/or received by the transducer array 154 may be disposed about the transducer array 154.

In FIG. 10, the transducer assembly 152 is shown as being disposed in a cavity 164 in the heart 162. As previously described with reference to FIG. 8, image data representative of an anatomical region of the patient 12 (see FIG. 1) may be acquired via an invasive probe having the exemplary transducer assembly illustrated in FIG. 6. The image data may be acquired in real-time employing the invasive probe. Alternatively, previously stored image data representative of the anatomical region may be acquired by the medical imaging system 18 (see FIG. 1). An image based on image data acquired via the invasive probe 14 (see FIG. 1) may be generated. The generated image may then be displayed on a display area, such as the display area 22 (see FIG. 1) on the medical imaging system 18. This acquisition of image data via the invasive probe aids a user in assessing need for therapy in the anatomical region being imaged.

Subsequently, one or more regions of interest requiring therapy may be identified on the displayed image. Reference numerals 166 and 170 are representative of a first region of interest and a second region of interest respectively. In certain embodiments, the user may visually identify the one or more regions of interest using the displayed image. Alternatively, in accordance with aspects of the present technique, tissue elasticity imaging techniques may be employed to aid the user in assessing the need for therapy in the one or more regions of interest. The tissue elasticity imaging techniques may include acoustic radiation force impulse (AFRI) imaging or vibroacoustography, for example. The transducer assembly 152 may be used to facilitate elasticity imaging. However, a separate dedicated array that is integrated onto the transducer assembly 152 may be utilized to achieve elasticity imaging.

Once the one or more regions of interest 166, 170 requiring therapy are identified, the medical imaging system 18 may be configured to facilitate delivery of therapy through the transducer assembly 152 to the identified regions of interest 166, 170. In one embodiment, the therapy may include high intensity focused ultrasound (HIFU) energy.

The medical imaging system 18 may deliver the therapy by steering an ablation beam towards an identified region of interest. Accordingly, in one embodiment, the ablation beam may include a steerable ablation beam. It should be noted that the ablation beam may be steered manually or electronically. The ablation beam may be steered using conventional phasing techniques that include phasing excitation of the ablation array to ensure propagation of the ultrasound beam in a desirable direction. It may be noted if the ablation beam is steerable, the one or more regions of interest 166, 170 within the field of view of the transducer assembly 152 may be ablated without repositioning the transducer assembly 152, thereby advantageously resulting in less movement of the transducer assembly 152 within the patient 12 (see FIG. 1). In other words, employing the transducer assembly 152 having a relatively wide field of view, the one or more regions of interest 166, 170 may be ablated while the transducer assembly 152 is positioned at a single location. For example, with the transducer assembly 152 positioned at a single location, a first ablation beam 168 may be steered towards the first region of interest 166, while a second ablation beam 172 may be steered towards the second region of interest 170. Subsequent to the ablation of the one or more regions of interest 166, 170, efficacy of ablation may also be assessed by imaging the ablated sites employing the transducer assembly 152.

By implementing the invasive probe as described hereinabove, a user may advantageously image a relatively wide field of view of an anatomical region employing the transducer assembly 152. Additionally, one or more regions of interest requiring therapy may be identified. Furthermore, employing the transducer assembly 152 that may also be configured to deliver therapy, the identified one or more regions of interest may be ablated within the relatively wide field of view of the transducer assembly 152. The transducer assembly 152 may also be utilized to facilitate assessment of efficacy of ablation performed employing a single device.

The various systems for imaging and providing therapy and method of imaging and providing therapy described hereinabove dramatically enhance efficiency of the process of imaging and delivering therapy, by integrating the imaging and therapy mapping aspects of the procedure. Employing the transducer assembly described hereinabove three-dimensional images having a relatively wide field of view may be generated from within a space-constrained environment, such as a catheter. In addition, use of relatively large transducer elements beneficially results in enhanced sensitivity. Further, as the transducer array is substantially stationary while the acoustically absorbing shell is configured to rotate with respect to the transducer array, torque requirements on the motor may be considerably reduced as motor is configured to rotate only the acoustically absorbing shell and not the interconnect.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A transducer assembly, comprising:
a cylindrical-shaped transducer array configured to transmit and receive acoustic energy over a three-dimensional volume; and
an acoustically absorbing shell disposed around the transducer array and configured to be rotatable with respect to the transducer array, wherein the acoustically absorbing shell is configured to selectively control directionality of acoustic energy transmitted and received by the transducer array.

2. The transducer assembly of claim 1, wherein the cylindrical-shaped transducer array comprises a plurality of ring-shaped transducer elements.

3. The transducer assembly of claim 2, wherein each of the plurality of ring-shaped transducer elements comprises a plurality of segments electrically coupled in series.

4. The transducer assembly of claim 1, wherein the transducer array comprises a lead zirconate titanate array, a micromachined ultrasound array or combinations thereof.

5. The transducer assembly of claim 1, wherein the each of the plurality of ring-shaped transducer elements comprises a piezocomposite material.

6. The transducer assembly of claim 1, wherein the transducer array is configured to be substantially stationary.

7. The transducer assembly of claim 1, wherein the acoustically absorbing shell comprises an acoustic window, and wherein the acoustic window is at least partially transparent to the acoustic energy.

8. The transducer assembly of claim 7, further comprising a motor in operative association with the acoustically absorbing shell and configured to rotate the acoustically absorbing shell with respect to the transducer array such that the acoustic window is oriented to selectively control the directionality of the energy.

9. The transducer assembly of claim 8, wherein the motor is configured to rotate the acoustically absorbing shell in a continuous mode, an oscillation mode or combinations thereof.

10. The transducer assembly of claim 7, wherein the acoustically absorbing shell comprises a focusing lens coupled to the acoustic window and configured to direct the transmission and reception of acoustic energy by the transducer array through the acoustic window.

11. The transducer assembly of claim 1, wherein the transducer assembly is configured for use in an invasive probe, and wherein the invasive probe comprises an imaging catheter, an endoscope, a laparoscope, a surgical probe, a transesophageal probe, a transvaginal probe, a transrectal probe, an intracavity probe, or a probe adapted for interventional procedures.

12. The transducer assembly of claim 1, further comprising acoustic coupling means disposed between the transducer array and the acoustically absorbing shell, wherein the acoustic coupling means is configured to couple the acoustic energy from the transducer array to the acoustically absorbing shell.

13. The transducer assembly of claim 1, further comprising a flexible interconnect layer, wherein the flexible interconnect layer comprises at least one conductive element disposed on a flexible substrate, and wherein the at least one conductive element is configured to facilitate coupling each of the plurality of transducer elements to a cable assembly or electronics.

14. An invasive probe configured to image an anatomical region, comprising:
an outer envelope sized and configured to be disposed in the anatomical region;
a transducer assembly disposed in the outer envelope, comprising:
a cylindrical-shaped transducer array configured to transmit and receive acoustic energy over a three-dimensional volume; and
an acoustically absorbing shell disposed around the transducer array and configured to be rotatable with respect to the transducer array, wherein the acoustically absorbing shell is configured to selectively control directionality of acoustic energy transmitted and received by the transducer array.

15. The invasive probe of claim 14, wherein the transducer array is configured to be substantially stationary with respect to the outer envelope.

16. The invasive probe of claim 14, wherein the acoustically absorbing shell comprises an acoustic window, and wherein the acoustic window is at least partially transparent to the energy.

17. The invasive probe of claim 16, further comprising a motor in operative association with the acoustically absorbing shell and configured to rotate the acoustically absorbing shell with respect to the transducer array such that the acoustic window is oriented to control the directionality of the energy.

18. The invasive probe of claim 17, wherein the motor is configured to rotate the acoustically absorbing shell in a continuous mode, an oscillation mode or combinations thereof.

19. The invasive probe of claim 17, wherein the motor is disposed within the invasive probe.

20. The invasive probe of claim 14, further comprising acoustic coupling means disposed between the transducer array and the acoustically absorbing shell, wherein the acoustic coupling means is configured to couple the acoustic energy from the transducer array to the acoustically absorbing shell.

21. The invasive probe of claim 14, further comprising acoustic coupling means disposed between the acoustically absorbing shell and the outer envelope, wherein the acoustic coupling means is configured to couple the acoustic energy from the acoustically absorbing shell to the outer envelope.

22. The invasive probe of claim 14, further comprising a position sensor configured to monitor a position of the acoustic window.

23. The invasive probe of claim 14, wherein the invasive probe comprises an imaging catheter, an endoscope, a laparoscope, a surgical probe, a transesophageal probe, a transvaginal probe, a transrectal probe, an intracavity probe, or a probe adapted for interventional procedures.

24. The invasive probe of claim 14, wherein the invasive probe is further configured to facilitate assessing the need for therapy in one or more regions of interest within the anatomical region and delivering therapy to the one or more regions of interest within the anatomical region.

25. A system, comprising:
an acquisition subsystem configured to acquire image data, wherein the acquisition subsystem comprises an invasive probe configured to image an anatomical region, wherein the invasive probe comprises:
an outer envelope sized and configured to be disposed in the anatomical region;
a transducer assembly disposed in the outer envelope, comprising:
a cylindrical-shaped transducer array configured to transmit and receive acoustic energy over a three-dimensional volume; and
an acoustically absorbing shell disposed around the transducer array and configured to be rotatable with respect to the transducer array, wherein the acoustically absorbing shell is configured to selectively control directionality of acoustic energy transmitted and received by the transducer array; and
a processing subsystem in operative association with the acquisition subsystem and configured to process the image data acquired via the acquisition subsystem.

26. The system of claim 25, wherein the acoustically absorbing shell comprises an acoustic window, and wherein the acoustic window is at least partially transparent to the energy.

27. The system of claim 26, further comprising a motor in operative association with the acoustically absorbing shell and configured to rotate the acoustically absorbing shell with respect to the transducer array such that the acoustic window is positioned to control the directionality of the energy.

28. The system of claim 25, wherein the processing subsystem comprises an imaging system, wherein the imaging system comprises an ultrasound imaging system, an optical coherence tomography system, or combinations thereof.

29. A method for imaging, comprising:
energizing a transducer array in a transducer assembly disposed in an invasive probe, wherein the transducer assembly comprises:
an acoustically absorbing shell disposed around the transducer array and configured to be rotatable with respect to the transducer array, wherein the acoustically absorbing shell comprises an acoustic window; and
selectively controlling directionality of acoustic energy transmitted by a portion of the transducer array aligned with the acoustic window.

30. The method of claim 29, wherein the step of selectively controlling comprises:
rotating the acoustically absorbing shell about the transducer array to orient the acoustic window at a plurality of positions;
transmitting acoustic energy generated by a portion of the transducer array aligned with a current position of the acoustic window; and
acquiring imaging data via the portion of transducer array aligned with the current position of the acoustic window.

31. The method of claim 30, further comprising generating an image from acquired image data for display on a display area of a medical imaging system.

32. The method of claim 31, wherein the step of generating an image comprises assembling imaging data acquired at each of the plurality of positions of the acoustically absorbing shell to obtain volumetric imaging data.

33. The method of claim 29, further comprising assessing need for therapy in one or more regions of interest within the anatomical region and delivering therapy to the one or more regions of interest within the anatomical region.
